(19) United States
(12) Patent Application Publication
(10) Pub. No.: US 2026/0023065 A1
(43) Pub. Date: Jan. 22, 2026

(54) Device and Method for Determining Inorganic Carbon System Parameters

(71) Applicants: Calcarea, Inc., Los Angeles, CA (US); Alphazeta Sciences LLC, Largo, FL (US)

(72) Inventors: Jess Firey Adkins, Altadena, CA (US); Sherwood Liu, Largo, FL (US)

(73) Assignees: Calcarea, Inc., Los Angeles, CA (US); Alphazeta Sciences LLC, Largo, FL (US)

(21) Appl. No.: 19/272,933

(22) Filed: Jul. 17, 2025

Related U.S. Application Data
(60) Provisional application No. 63/672,492, filed on Jul. 17, 2024.

(51) Int. Cl. G01N 33/18; G01N 31/22

(52) U.S. Cl. CPC G01N 33/1893; G01N 31/223

(57) ABSTRACT

A device and method for a differential analysis of ocean's CO₂ are described. In particular, the device and method allow quantification of CO₂ removal and storage in seawater (or freshwater), wherein the device and method measure the difference in the three parameters of the aqueous inorganic carbon system—pH, Dissolved Inorganic Carbon (DIC), and Total Alkalinity (TA)—for a pair of samples comprising an untreated sample of ambient seawater (or another water source) and a treated sample comprising an effluent leaving a CO₂ removal apparatus, to determine a bicarbonate concentration difference between the treated and untreated samples and, as such, the amount of CO₂ absorbed via the CO₂ removal apparatus.

### CROSS-REFERENCE TO RELATED APPLICATIONS

This application claims the benefit of priority under 35 U.S.C. § 120 to U.S. Patent Application No. 63/672,492, filed on Jul. 17, 2024, the disclosure of which is hereby incorporated by reference herein in its entirety.

### FIELD OF THE INVENTION

The current disclosure is directed to a device for a differential analysis of ocean's CO₂ and a method of use thereof.

### BACKGROUND OF THE INVENTION

Carbon dioxide (CO₂) constitutes about 0.04% (400 parts per million) of the atmosphere. Despite its relatively small overall concentration, CO₂ is a potent greenhouse gas that plays an important role in regulating the Earth's surface temperature. Presently, anthropogenic CO₂ generation is taking place at a rate greater than it is being consumed and/or stored, leading to increasing concentrations of CO₂ in the atmosphere. There is a growing concern that rising levels of CO₂ in the earth's atmosphere may present a substantial environmental challenge. As a result, there is an increased interest in developing methods for removing CO₂ from emission streams and the atmosphere and storing it in a manner that prevents its future release into the atmosphere. This capture and storage are collectively known as CO₂ sequestration. As such, carbon capture and storage (CCS) efforts have long been centered on Earth's atmosphere, with companies scrubbing carbon dioxide from air or directly from polluter's exhaust and storing it underground or in the ocean. In particular, a number of promising methods for sequestration and permanent storage of CO₂ in the ocean as bicarbonate have been reported (see for examples: U.S. Pat. Nos. 10,920,249; 11,235,278; U.S. patent application Ser. No. 18/645,286; U.S. Pat. Nos. 6,890,497; and 11,629,067, the disclosures of which are incorporated herein by reference). However, the quantitative evaluation of the efficacy of CO₂ removal via ocean storage approaches is less straightforward than with land-based methods, and, as such, methods and technology for accurately and precisely measuring the effectiveness of the ocean storage methods are lacking, thus hindering widespread implementation of the CO₂ ocean storage methods.

In addition, measurements of four seawater inorganic carbon system parameters, i.e., pH, partial pressure of CO₂ (pCO₂), total dissolved inorganic carbon (DIC) or CT (total carbon), and total alkalinity (TA), are essential for carbon cycle investigations on both global and local scales. To this end, both observational and modeling efforts rely on high quality inorganic carbon data from field measurements. It should be noted here, that while only two of these four parameters are needed to determine the seawater inorganic carbon system, having access to more than two parameters provides internal checks, as the system becomes an overdetermined system. As such, extensive efforts have been devoted to improving methodologies and instruments for determination of carbon parameters in seawater. Accordingly, there exists a great and urgent need for precise and accurate, yet practical, solutions to straightforwardly accessing a suite of seawater inorganic carbon system parameters.

### SUMMARY OF THE INVENTION

Various embodiments are directed to a device for determining inorganic carbon system parameters for a pair of aqueous samples including:
an enclosure characterized by a footprint size further including:
   a dry electronics chamber including a power supply, a computer, a display, a light source, a relay board, a connectivity hub, a square wave generator for pumps, a pH spectrometer, a DIC spectrometer, and a TA spectrometer;
   a pumps and valves chamber including a plurality of diaphragm pumps further including a sample pump, a pH channel pump, a pH dye pump, a DIC channel pump, a DIC reference pump, a DIC dye pump, a DIC standard solution pump, TA channel pump, and a TA dye pump; and a plurality of valves further including: a sample selection valve, wherein the sample selection valve is a 3-way valve; a sample outlet, a 3-way TA sample valve, a 3-way CO₂ valve, and a 2-way DIC LCW shutoff valve;
   a reagents chamber including a pH dye reservoir containing a pH dye; a DIC reservoir containing DIC reagents, including a DIC reference, and a DIC dye; a TA dye reservoir containing a TA dye; and a DIC acid reservoir containing a DIC acid;
   a sample reservoir and
   a wet chamber characterized by a temperature controlled by a temperature controller, further including
      a pH channel including a pH optical cell in optical communication with the pH spectrometer and the light source, in thermal contact with the temperature controller, and in fluid communication with the sample selection valve, the sample outlet, and the pH dye reservoir;
      a DIC channel including a DIC optical cell in optical communication with the DIC spectrometer and the light source, in thermal contact with the temperature controller, and in fluid communication with the sample selection valve, the sample outlet, the DIC acid reservoir, and the DIC reservoir;
      a TA channel including a TA optical cell in optical communication with the TA spectrometer and the light source, in thermal contact with the temperature controller, and in fluid communication with the sample selection valve, the sample outlet, the TA dye reservoir, and a TA CO₂ supply of a known CO₂ concentration; and wherein
the temperature controller is disposed in an immediate proximity to the wet chamber to control the temperature.

In various such embodiments, the wet chamber further includes at least one or all of: a salinity sensor, a temperature sensor, and a stirring device to promote water circulation within the wet chamber.

In still various such embodiments, the temperature controller is a Peltier thermostat.

In still yet various embodiments, the light source is an LED.

In yet still various such embodiments, the connectivity hub is a USB hub.

In yet various such embodiments, the DIC optical cell includes an LCW tubing enclosed within an outer PEEK tubing, such that the LCW tubing is in optical communication with the DIC spectrometer and the light source, and in fluid communication with the DIC reagent reservoir; while the outer PEEK tubing is in fluid communication with the sample selection valve, the sample outlet, and the DIC acid reservoir, such that the outer PEEK tubing contains a solution that is in diffusive communication with the LCW tubing.

In various such embodiments, the LCW tubing is gas permeable, optically clear and is characterized by a refractive index of near 1.29 or less.

In still various such embodiments, the LCW tubing includes Teflon AF.

In yet still various such embodiments, the TA channel includes the TA optical cell enclosed within an outer TA tubing, such that the TA optical cell is in optical communication with the TA spectrometer and the light source; and in fluid communication with the sample selection valve, the TA dye reservoir, and the CO₂ supply; and wherein the outer TA tubing is in fluid communication with the CO₂ supply.

In still yet various such embodiments, the known CO₂ concentration of the CO₂ supply to the TA channel is 8 to 20%.

In yet various such embodiments, the known CO₂ concentration is 10%.

Various other embodiments are directed to a method for determining inorganic carbon system parameters including:
providing a device including:
   an enclosure characterized by a footprint size further including:
      a dry electronics chamber including a power supply, a computer, a display, a light source, a relay board, a connectivity hub, a square wave generator for pumps, a pH spectrometer, a DIC spectrometer, and a TA spectrometer;
      a pumps and valves chamber including a plurality of diaphragm pumps further including a sample pump, a pH channel pump, a pH dye pump, a DIC channel pump, a DIC reference pump, a DIC dye pump, a DIC standard solution pump, TA channel pump, and a TA dye pump; and a plurality of valves further including: a sample selection valve, wherein the sample selection valve is a 3-way valve; a sample outlet, a 3-way TA sample valve, a 3-way CO₂ valve, and a 2-way DIC LCW shutoff valve;
      a reagents chamber including a pH dye reservoir containing a pH dye; a DIC reservoir containing DIC reagents, including a DIC reference and a DIC dye; a TA dye reservoir containing a TA dye; and a DIC acid reservoir containing a DIC acid;
      and a sample reservoir; and
      a wet chamber characterized by a temperature controlled by a temperature controller, further including
         a pH channel including a pH optical cell in optical communication with the pH spectrometer and the light source, in thermal contact with the temperature controller, and in fluid communication with the sample selection valve, the sample outlet, and the pH dye reservoir;
         a DIC channel including a DIC optical cell in optical communication with the DIC spectrometer and the light source, in thermal contact with the temperature controller, and in fluid communication with the sample selection valve, the sample outlet, the DIC reservoir, and the DIC acid reservoir;
         a TA channel including a TA optical cell in optical communication with the TA spectrometer and the light source, in thermal contact with the temperature controller, and in fluid communication with the sample selection valve, the sample outlet, the TA dye reservoir, and a TA CO₂ supply of a known CO₂ concentration; and wherein
      the temperature controller is disposed in an immediate proximity to the wet chamber to control the temperature;
   providing a pair of different aqueous samples including a first sample and a second sample;
   splitting the first and the second samples into three aliquots each to obtain a first through a third aliquot of the first sample and a first through a third aliquot of the second sample;
   feeding the first aliquot of the first sample through the sample selection valve to one of: the pH channel, the DIC channel, or the TA channel;
   measuring a first parameter, wherein the first parameter is a parameter corresponding to the selected channel, that is a first pH, a first DIC, or a first TA, and
   purging the first aliquot of the first sample from the device through the sample outlet;
   feeding the first aliquot of the second sample through the sample selection valve to the same channel,
   measuring a second parameter, wherein the second parameter is a parameter corresponding to the selected channel, that is a second pH, a second DIC, or a second TA, and
   purging the first aliquot of the second sample from the device through the sample outlet;
   recording the first parameter, the second parameter, and
   calculating the difference between the first parameter and the second parameter;
   repeating feeding, measuring, purging, recording, and calculating steps for the remaining aliquots for the remaining channels,
to completely characterize the inorganic carbon system parameters of the first and the second samples.

In various such embodiments, the wet chamber further includes at least one or all of: a salinity sensor, a temperature sensor, and a stirring device to promote water circulation within the wet chamber.

In still various such embodiments, the temperature controller is a Peltier thermostat.

In still yet various embodiments, the light source is an LED.

In yet still various such embodiments, the aqueous component of the pair of different aqueous samples is seawater.

In yet various such embodiments, the aqueous component of the pair of different aqueous samples is freshwater.

In various such embodiments, the DIC optical cell includes an LCW tubing enclosed within an outer PEEK tubing, such that the LCW tubing is in optical communication with the DIC spectrometer and the light source, and in fluid communication with the DIC reagent reservoir; while the outer PEEK tubing is in fluid communication with the sample selection valve, the sample outlet, and the DIC acid reservoir, such that the outer PEEK tubing contains a solution that is in diffusive communication with the LCW tubing.

In still various such embodiments, the LCW tubing is gas permeable, optically clear and is characterized by a refractive index of near 1.29 or less.

In yet still various such embodiments, the TA channel includes the TA optical cell enclosed within an outer TA tubing, such that the TA optical cell is in optical communication with the TA spectrometer and the light source; and in fluid communication with the sample selection valve, the TA dye reservoir, and the CO₂ supply; and wherein the outer TA tubing is in fluid communication with the CO₂ supply.

In still yet various such embodiments, the known CO₂ concentration of the CO₂ supply to the TA channel is 10%.

In yet various such embodiments, measuring the first and the second DIC, each, includes:
flushing the outer PEEK tubing with the first or second sample;
acidifying the first or second sample with the DIC acid;
flushing the LCW tubing with the DIC reference;
taking a reference measurement;
pumping the DIC dye solution of known alkalinity into the LCW tubing; and
   allowing to equilibrate prior to taking a measurement.

In various such embodiments, measuring the first and the second TA, each, includes:
flushing the TA optical cell with the first or second sample;
taking a reference measurement;
pushing a plug of the TA dye in the TA channel loop just prior to the optical cell;
using the CO₂ supply to carry the TA dye into the TA optical cell, and
   allowing to equilibrate with the CO₂ gas in the outer PEEK tubing prior to taking a measurement.

Additional embodiments and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which forms a part of this disclosure.

### BRIEF DESCRIPTION OF THE DRAWINGS

These and other features and advantages of the present invention will be better understood by reference to the following detailed description when considered in conjunction with the accompanying data and figures, wherein:

FIGS. 1A and 1B provide and compare the result of calculating DIC, Alkalinity, and [HCO₃⁻] using pH and pCO₂ parameters (FIG. 1A) according to prior art; or DIC, pCO₂, and [HCO₃] using pH and Alkalinity parameters (FIG. 1B) in accordance with embodiments of the application.

FIGS. 2A and 2B schematically illustrate the hardware layout of the device for CO₂ removal analysis, in accordance with embodiments of the application

FIG. 3 schematically illustrates the method in accordance with embodiments of the application.

FIG. 4 schematically illustrates the pH channel of the device, in accordance with embodiments of the application.

FIGS. 5A and 5B illustrate the principles of spectrophotometric pH measurement employed in the device and method, with FIG. 5A more specifically providing pH dependent spectra of m-cresol purple, according to prior art.

FIG. 6 schematically illustrates the DIC channel of the device, in accordance with embodiments of the application.

FIG. 7 schematically illustrates properties of Teflon® AF liquid core waveguide (LCW) according to prior art.

FIG. 8 illustrates the timing of DIC analysis within the DIC channel in accordance with embodiments of the application.

FIG. 9 provides illustrative data collected via Bayboro Harbor in situ DIC measurements (top) and SHARQ in situ DIC measurements, according to prior art.

FIG. 10 schematically illustrates the TA channel of the device, in accordance with embodiments of the application.

FIGS. 11A through 11C illustrate TA equilibration analysis with the TA channel, including illustrating that at constant salinity and temperature, AT is a simple relationship to R ratio (FIGS. 11B and 11C), in accordance with embodiments of the application.

FIG. 12 provides data comparing titration-based methods according to prior art and the TA equilibration-based method in accordance with embodiments of the application.

FIG. 13 provides illustrative data collected by the device and method over a month at sea in the pH and DIC channels, in accordance with embodiments of the application.

### DETAILED DISCLOSURE

The embodiments of the invention described herein are not intended to be exhaustive or to limit the invention to precise forms disclosed. Rather, the embodiments selected for description have been chosen to enable one skilled in the art to practice the invention.

Turning to the drawings, schemes, and data, embodiments of a device for quantifying the effectiveness of CO₂ removal and storage in seawater and a method of use thereof are provided. In many embodiments, the device and method continuously measure the difference in the three parameters of the seawater inorganic carbon system—pH, Dissolved Inorganic Carbon (DIC), and Total Alkalinity (TA)—for a pair of seawater samples taking turns in passing through the device, wherein the pair of seawater samples comprises an untreated sample, comprising ambient water (e.g. seawater), and a treated sample, comprising an effluent leaving a CO₂ removal apparatus, to determine a bicarbonate concentration difference between the treated and untreated samples and, as such, the amount of CO₂ captured and permanently stored by the CO₂ removal apparatus. Accordingly, in many embodiments, the CO₂ removal efficacy of a CO₂ removal apparatus is calculated by the method as a combination of two of either a DIC change, a pH change, or an alkalinity change in the treated sample, as compared to the untreated sample; wherein the total CO₂ removal is then calculated for the known volume of water treated by the CO₂ removal apparatus. In many embodiments, a Peltier control board/thermostat is employed for the device's temperature control. In many embodiments, the device and method's design and choice of components allow for miniaturization of the device. In many embodiments, the device and method are fully automated. In many embodiments, the device and method are employed in a Carbon Dioxide Removal (CDR) application, such as, for example, a shipboard underway system for CO₂ capture and storage in the ocean. In some embodiments, the device and method are employed in high quality inorganic carbon data collection for any kind of oceanic research. However, in some embodiments, the device is adjusted for use with freshwater samples, such as, for example, water samples from lakes, rivers, streams, ground water, ponds, aquifers, and wells.

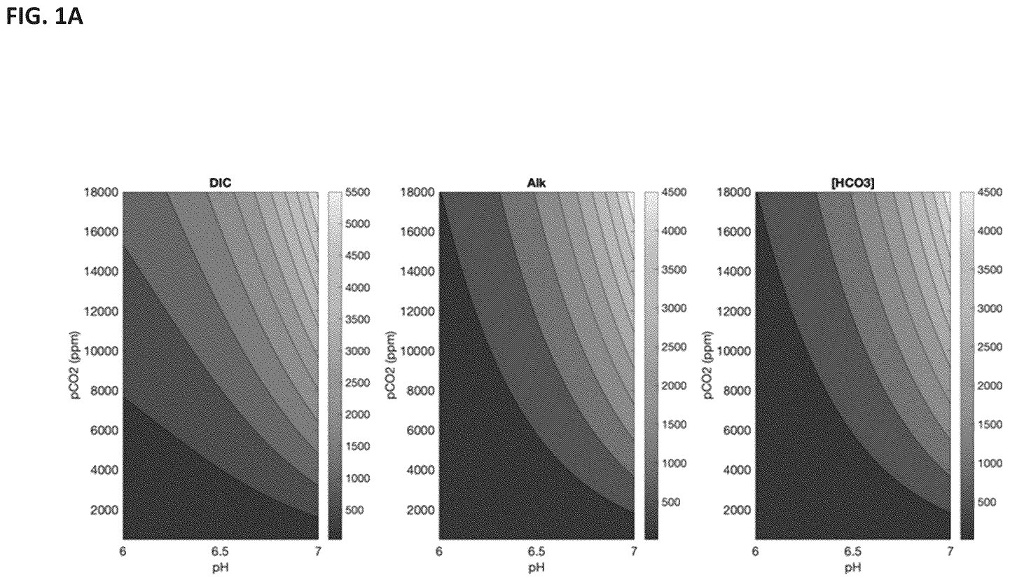

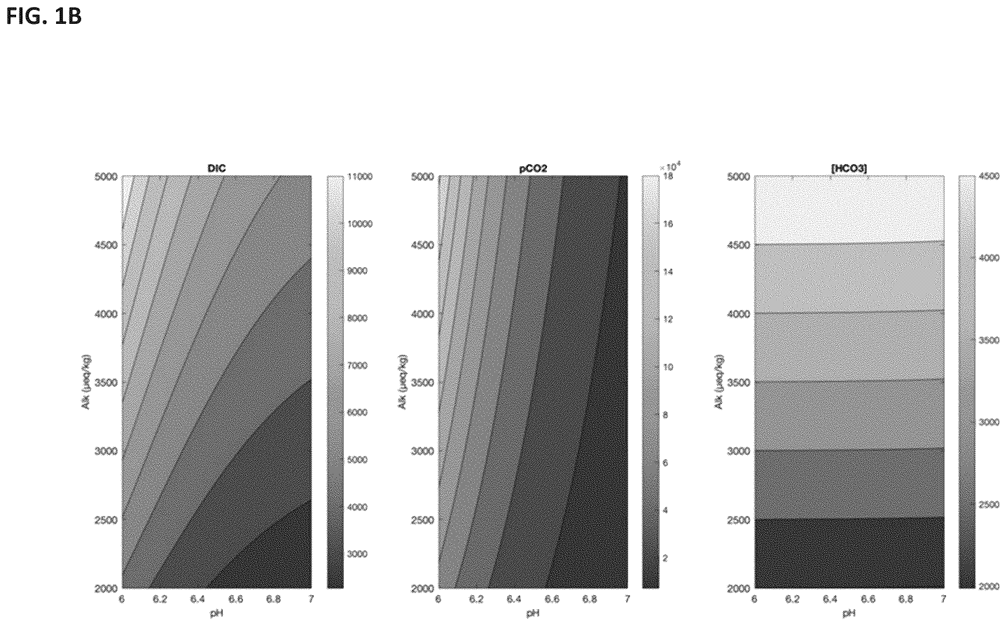

Accordingly, in many embodiments, the device collects data to afford three types of output measurements for samples of seawater—pH, DIC, and TA. In many embodiments, these three measurements allow to solve the seawater inorganic carbon system for the samples, and, as such, to determine concentrations of carbonic acid ([H₂CO₃]), as well as bicarbonate ([HCO₃]) and carbonate ([CO₃²⁻]) ions concentrations in such samples (FIG. 1B). In particular, bicarbonate concentration is the key metric for documenting safe and permanent storage of carbon in the ocean via any process that converts CO₂ into the soluble forms using a base.

In many embodiments, the device and method described herein allow for a robust, accurate, and precise determination of pH, DIC, and TA differences for pairs of different seawater samples and, as such, a more precise characterization of the differences in the seawater inorganic carbon system for such pairs of samples than any other device employed for a similar purpose to date. For example, FIG. 1A shows the result of calculating DIC and [HCO₃⁻] changes in seawater samples using data collected by a Multiparameter Inorganic Carbon Analyzer (MICA) device (described in U.S. Pat. No. 8,077,311, the disclosure of which is incorporated herein by reference), wherein partial pressure of CO₂ (pCO₂) is measured along with pH and DIC; while FIG. 1B shows the result of calculating the same using data collected by the device and method of many embodiments, wherein Total Alkalinity (TA) is measured along with pH and DIC. It should be noted here that, in both of these examples, pH represents the master variable, because it is the most sensitive parameter for calculating the various species of the inorganic carbon system (e.g., [CO₃²⁻] and pCO₂), and is the basis of the sensor dye's variation in light absorbance. As can be seen from these figures, using Alkalinity instead of pCO₂ leads to a very simple structure of the changes in [HCO₃⁻]. In fact, according to many embodiments, for a given measurement uncertainty in pH, Alkalinity, and pCO₂, the calculated [HCO₃⁻] is more precise when Alkalinity and pH data is used, than when pCO₂ and pH data is used. Furthermore, the [HCO₃⁻] is not strongly dependent on pH in the range of values expected for the effluent leaving a CO₂ removal apparatus (i.e., the treated sample).

In many embodiments, according to the method, the measurements afforded by the device are all conducted for the difference between the untreated and treated samples, thus eliminating some of the measurements needed in conventional devices, or, at least, their needed precision and accuracy. In other words, in many embodiments, the method relies on the parameters' differences between the untreated sample and treated sample measured by the device to calculate the additional bicarbonate ion concentration resulting from CO₂ capture (or any other inorganic carbon system concentration). As such, since there is no need to calculate the absolute values for any of the measured parameters for either sample of the pair of samples, several parts of the relevant calculation math cancel out. Furthermore, since it can be assumed that the salinity of the samples is the same at the necessary precision, such math cancelations include the salinity dependent terms and, therefore, provide a much less stringent salinity specifications for the instrument. In many embodiments, this feature affords a much simpler and less expensive salinometers and contributes to the robustness of the data provided by the device.

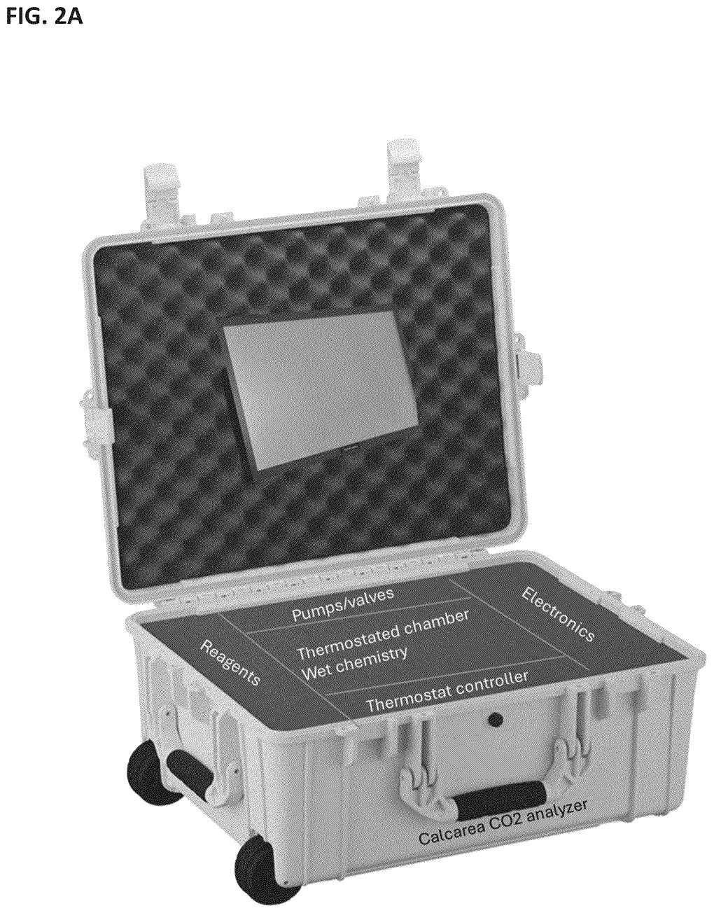

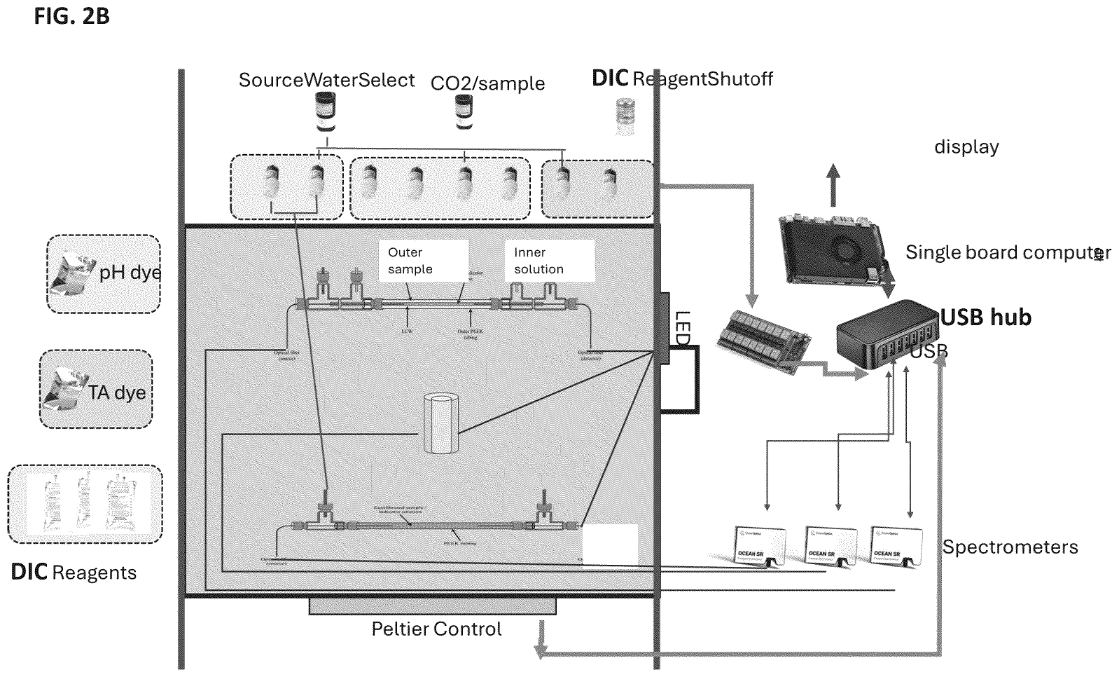

FIGS. 2A and 2B schematically illustrate the device and method of the instant application. In particular, as shown in FIG. 2A, and according to many embodiments, the device comprises an enclosure further comprising: a wet chamber for wet chemistry characterized by a temperature, wherein the temperature is controlled by a temperature controller; a dry electronics chamber; a pumps and valves chamber; and a reagents chamber. In many embodiments, the temperature controller is a Peltier control board/thermostat. In many embodiments, the Peltier control board is disposed in an immediate proximity to the wet chamber, such as, for example, underneath and directly adjacent to the wet chamber, as shown in FIGS. 2A and 2B. In many embodiments precautions are taken to vent the enclosure in the proximity of the Peltier control board. To this end, in many embodiments the device comprises at least two height adjustable legs to raise the device up for air circulation. In many embodiments, the enclosure has a small footprint of less than 6×2 ft². In many embodiments, the enclosure's footprint is 2×1.5 ft² or less. In many embodiments, the enclosure is 22×17×11 inches.

Furthermore, as more specifically shown in FIG. 2B, in many embodiments, the wet chamber comprises three channels, wherein each channel further comprises an optical cell in communication with an appropriate spectrometer and a light source via an optical fiber; in thermal contact with the temperature controller; and in fluid communication with a sample source (via a sample valve), a sample outlet, and an appropriate reagent reservoir containing an appropriate reagent. More specifically, in many such embodiments, the wet chamber comprises: a pH channel comprising a pH optical cell in optical communication with a pH spectrometer and the light source; in thermal contact with the temperature controller; and in fluid communication with the sample valve, the sample outlet, and a pH dye reservoir comprising a pH dye; a DIC channel comprising a DIC optical cell in optical communication with a DIC spectrometer and the light source; in thermal contact with the temperature controller; and in fluid communication with the sample valve, the sample outlet, a DIC reagent reservoir comprising DIC reagents, and a DIC acid reservoir comprising an acid for acidifying the sample entering the DIC channel; and a TA channel comprising a TA optical cell in optical communication with a TA spectrometer and the light source; in thermal contact with the temperature controller; and in fluid communication with the sample valve, the sample outlet, and a TA dye reservoir comprising a TA dye. In many embodiments, the TA channel is connected to a CO₂ gas reservoir as a TA reagent reservoir. In addition, in many embodiments, the wet chamber further comprises a salinity sensor and a temperature sensor. Moreover, in many embodiments, the wet chamber comprises a stirring device to promote water circulation within the wet chamber.

In many embodiments, the temperature control over the device's operation is provided by the Peltier control board. This is in contrast to many conventional devices cooled by a water bath. In many such embodiments, the implementation of the Peltier thermostat for temperature control, instead of a more conventional use of a water bath, removes the ‘wetness’ factor from the device's design, and, also, allows for miniaturization of the device. In many embodiments, Peltier control board allows for temperature control of the wet chamber with 0.02° C. precision without the need to contain a water bath inside the device.

In many embodiments, the dry electronic chamber at least comprises a power supply, a computer, a display, a light source, such as, for example an LED, a relay board, a connectivity hub, such as, for example, a USB hub, a square wave generator for pumps, the pH spectrometer, the DIC spectrometer, and the TA spectrometer (FIG. 2B). In many such embodiments, the power supply provides as little as 200 w of power, which is in contrast to conventional devices requiring as much as 1,000 w or more. In many embodiments, the light source is a separate wide spectrum LED. In contrast, conventional state of the art devices typically employ a desktop computer with the relay board and the light source (e.g., Tungsten 4×) built in.

Furthermore, in many embodiments, the pumps and valves chamber comprises a plurality of pumps and valves as shown in FIG. 2B. In many embodiments, the plurality of pumps is the plurality of diaphragm pumps, rather than conventionally used peristatic pumps. This is an important distinction because, in contrast to the diaphragm pumps, peristaltic pumps require flexible tubing to operate, wherein such tubing is known to degas CO₂ and, as such, change the DIC and pH values. Furthermore, in many embodiments, the plurality of pumps comprises: a sample pump, a pH channel pump, a pH dye pump, a DIC channel pump, a DIC reference pump, a DIC dye pump, a DIC standard solution pump, a DIC acid pump, a TA channel pump, and a TA dye pump. In many embodiments, the plurality of valves comprises: a sample selection valve (3 ways), a TA sample/CO₂ valve (3 ways), a DIC liquid core waveguide (LCW) shutoff valve (2 ways). In many embodiments, a sample is introduced to the wet chamber through a pass-through connector which is connected to the sample selector valve.

Moreover, in many embodiments, the reagent chamber comprises various reagents for the device's three measurements/channels, wherein the reagents are disposed within the corresponding to the measurement types reagent reservoirs in fluid communication with the corresponding wet chamber channels (FIG. 2B). In many embodiments, the reagent chamber comprises: the pH dye reservoir comprising the pH dye, such as, for example, m-cresol purple (mCP) or phenol red; the DIC reservoir comprising the DIC reagents, including a DIC reference, and a DIC dye bromocresol purple (BCP); and the TA dye reservoir comprising the TA dye, such as, for example, also bromocresol purple (BCP). In addition, in many embodiments, the reagents chamber also comprises a DIC acid (such as, for example, 2N HCl), such that it can be added to the sample prior to entering the DIC channel to acidify the sample.

In many embodiments, the device further comprises a de-bubbling device disposed within the sample line, such as to eliminate any bubbles in the light path that might affect the pH and DIC measurements. In addition, in many embodiments, the cell is periodically rinsed with a dilute acid to prevent fouling that might affect the pH measurements.

In many embodiments, the device is also equipped with the Global Positioning System (GPS), allowing for the collected data to be enhanced with the location that the seawater sample was collected from, including a time and latitude/longitude stamp for every collected sample.

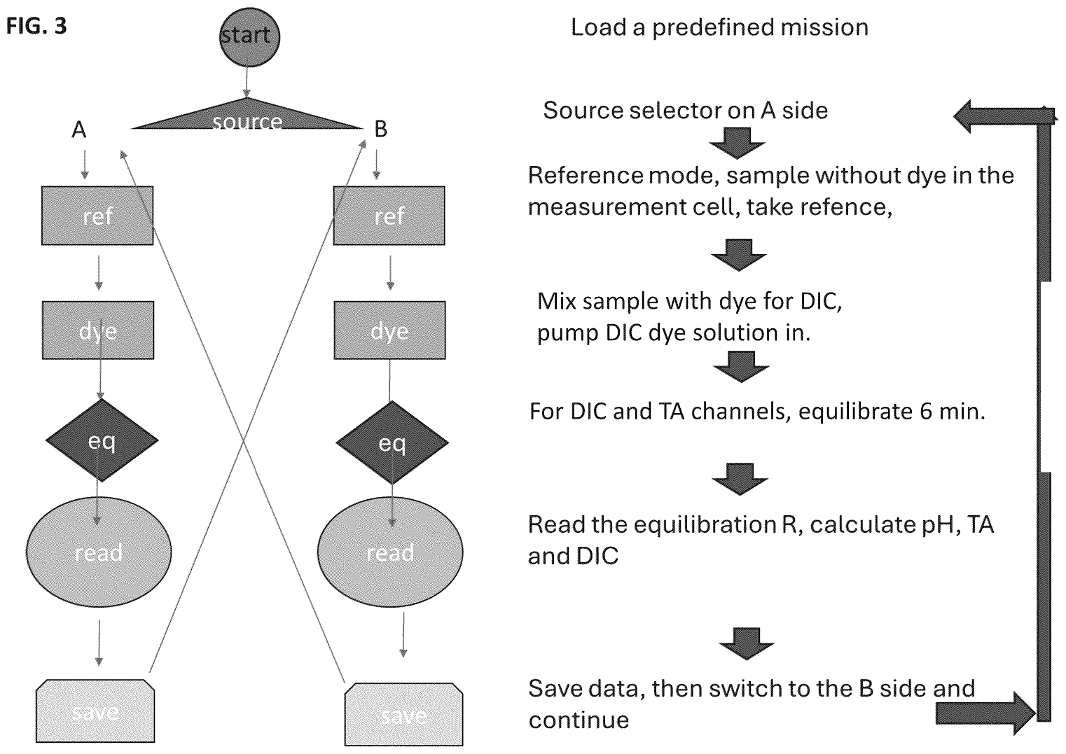

To this end, in many embodiments, the pair of seawater samples comprising the untreated (ambient) and treated samples is collected and delivered to the device. Next, the untreated and treated samples, A and B sides of the diagram in FIG. 3, respectively, are sequentially fed into the device via the sample selection valve in three aliquots each, wherein each of the three aliquots is delivered to one of: the pH, the DIC, and the TA channels of the wet chamber, for the corresponding parameter measurement as described below. To this end, as schematically illustrated in FIG. 3, the device is first loaded with a predefined mission—the type of the measurement/channel an aliquot is destined for, followed by selection of that aliquot's source (i.e., source A for the untreated sample and source B for the treated sample). Next, the aliquot is pumped into the channel of choice for the predefined mission with that channel in a reference mode, and a reference measurement of the aliquot is made. Next, the dye or reagent solution corresponding to the desired measurement is pumped into the channel of the mission, mixed with the sample's aliquot therewithin, and allowed to equilibrate (for as long as 6 minutes for DIC and TA measurements), before all the reference and sample measurements are recorded, and the parameter of choice (i.e., pH, TA or DIC) calculated. Next, the aliquot exits the device through the sample outlet, and the obtained and calculated data is saved. Still next, the measurement process of the instant method is switched to the other source of aliquots (i.e., source B if source A was used first), and the same parameter is measured for the newly sourced aliquot. Next, the same sequence is followed with the rest of the aliquots to obtain a full set of pH, DIC, and TA measurements, as well as the corresponding value differences, for each pair of collected seawater samples. In many embodiments, the device and method afford measurements every 5-6 minutes per parameter, and 10-12 minutes for the paired (unprocessed/processed) samples.

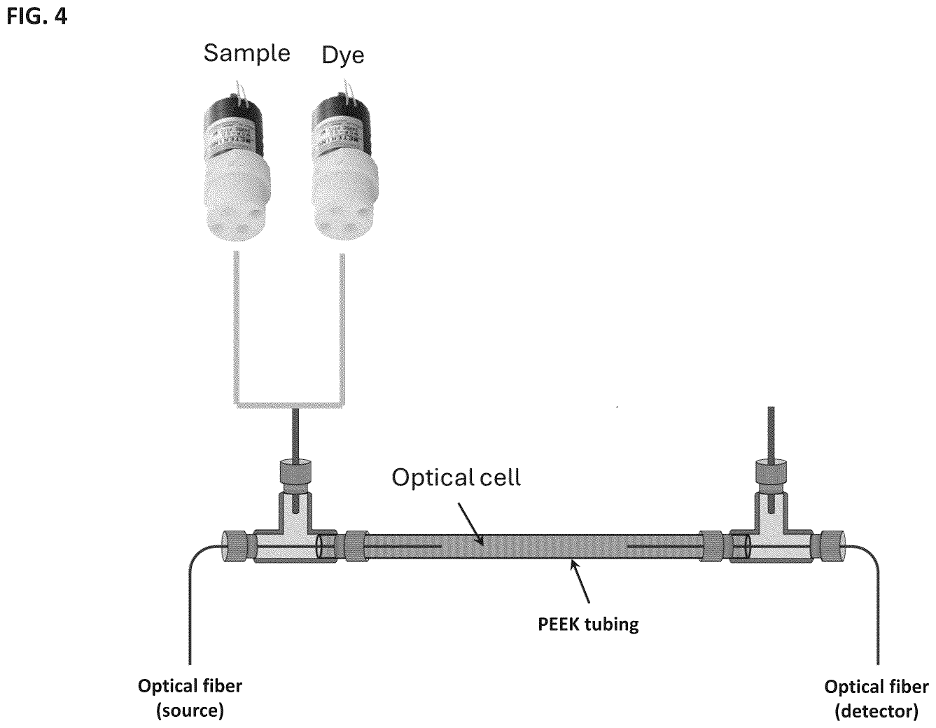

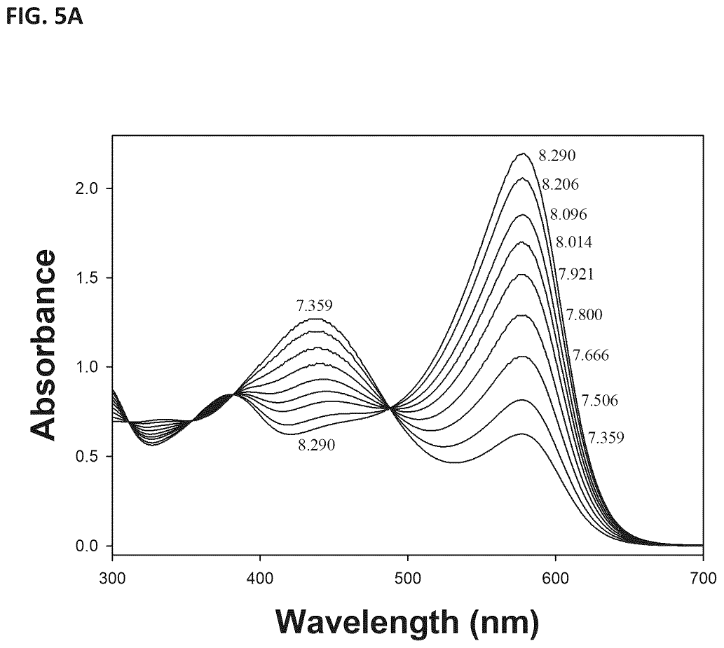

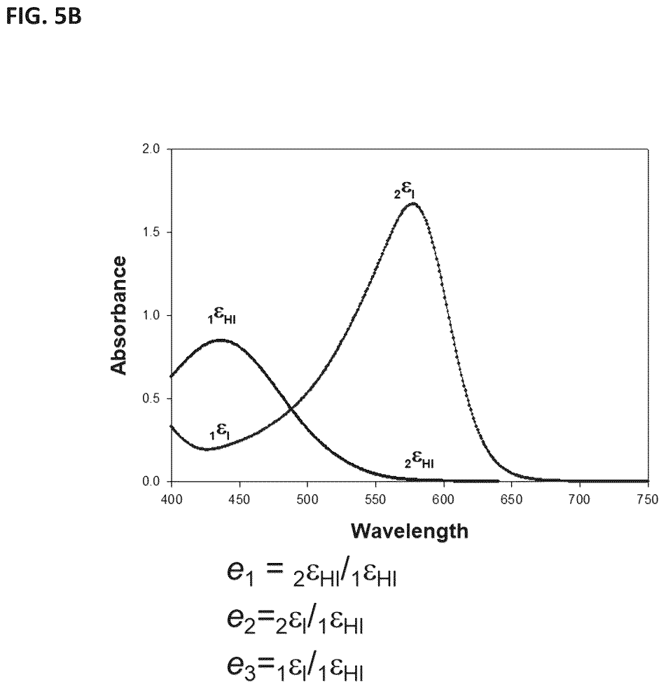

Furthermore, FIG. 4 schematically shows the pH channel of many embodiments employed in the device and method of the instant application for pH measurements, while FIGS. 5A and 5B provide exemplary data illustrating the principles of such spectrophotometric pH measurements, wherein:

$${HI}^{-} ↔ {H}^{+} + {I}^{2 -}$$

$${K}_{2} = {[ {I}^{2 -} ] [ {H}^{+} ] [ {HI}^{-} ]}^{- 1}$$

$$pH = {pK}_{2} + log ( \frac{R - {e}_{1}}{{e}_{2} - {Re}_{3}} ) .$$

More specifically, here, FIG. 5A provides illustrative spectra of pH dependent m-Cresol Purple dye, which is used as the pH dye in the pH measurements of many embodiments. As seen from FIG. 4, and according to many embodiments, the pH channel comprises the pH optical cell comprising polyetheretherketone (PEEK) tubing in optical communication with the light source and the pH spectrometer via an optical fiber, as well as in fluid communication with the sample selection valve and the pH dye reservoir.

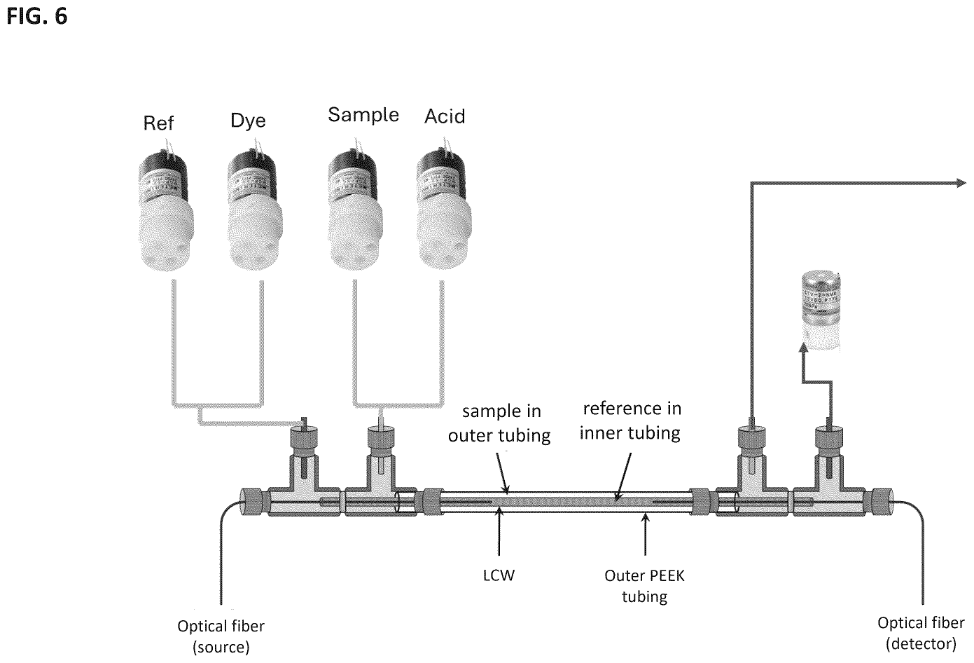

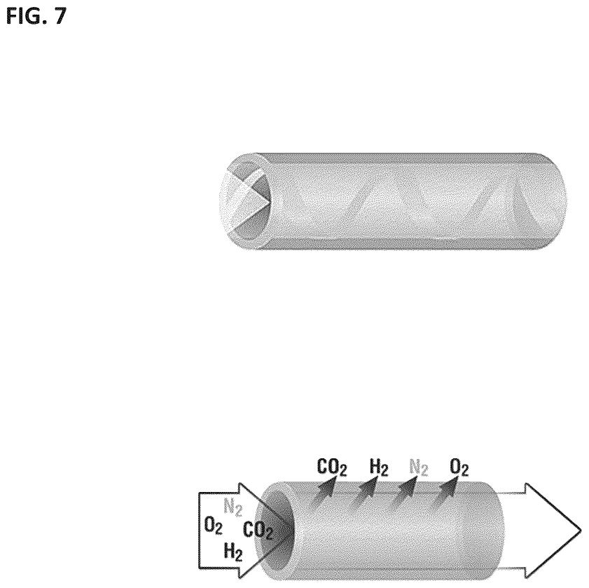

Next, FIG. 6 schematically shows the DIC channel of many embodiments employed in the device and method of the instant application for DIC measurements. This design follows the work of Byrne et al. (2002), Analytica Chimica Acta, 451, 221-229, (the disclosure of which is incorporated herein by reference), wherein a seawater sample is first acidified to convert all inorganic carbon species into CO₂* (the combination of CO₂ (aq) and H₂CO₃), such that the amount of CO₂* can be measured by equilibrating this acidified solution with a solution of known alkalinity, followed by measuring the pH after the equilibration. To this end, in many embodiments, illustrated in FIG. 6, the DIC channel comprises the DIC optical cell further comprising a liquid core waveguide (LCW) tubing enclosed within an outer PEEK tubing; wherein the LCW tubing is in optical communication with the light source and the DIC spectrometer via an optical fiber; and in fluid communication with the sample selection valve and the DIC reagents reservoir (comprising the DIC reference and the DIC dye); and further wherein the outer PEEK tubing is in fluid communication with the sample reservoir and the DIC acid reservoir, so that the outer PEEK tubing contains a solution that is in diffusive communication with the inner tubing for transfer of CO₂* supply from the acidified sample (i.e., aqueous CO₂ resulting from sample acidification with the DIC acid). In many embodiments, the LCW tubing is so thin as to allow gas exchange across its walls. In many embodiments, the LCW tubing comprises Teflon AF 2400. In many embodiments, the LCW tubing allows CO₂ equilibration across its wall, acting as a membrane. More specifically, Teflon® AF serving as liquid core waveguide offers excellent optical clarity and extremely low refractive index (Teflon® AF Index of Refraction is 1.29, wherein H₂O Index of Refraction is 1.33), wherein Teflon® AF tubing forms an optical fiber when filled with virtually any transparent liquid, including water (FIG. 7, top). Furthermore, Teflon® AF provides fast gas removal (FIG. 7, bottom), thus offering the combination of exceptional permeability and outstanding chemical and solvent resistance, which makes Teflon® AF the preferred material in manufacturing highly efficient degassing devices.

As such, in many embodiments, the DIC channel shown in FIG. 6, allows for DIC quantification according to the following equations, given that B(t) is an empirically determined constant that is a function of temperature, salinity, and (temperature):

$$log DIC = log ( \frac{{( {K}_{0} )}_{a}}{{( {K}_{0} )}_{i}} ) + B - log ( \frac{R - {e}_{1}}{1 - {Re}_{3} / {e}_{2}} ) .$$

$$log DIC = log ( \frac{{( {K}_{0} )}_{a}}{{( {K}_{0} )}_{i}} ) + B + pH .$$

Here, the equilibrium constants (K₀) are the ‘Henry's Law’ constants for CO₂ exchange and, as such, comprise the values for the acidified external solution (labeled “a”) and the solution in the internal wave guide (labeled “i”). Therefore, in many embodiments, the procedure to measure DIC with the DIC channel of many embodiments comprises:
acidifying seawater outside the LCW tubing with the DIC acid to convert all CO₂ to [CO₂*];
flushing reference solution inside the LCW tubing;
taking a reference measurement;
pumping internal dye solution of known alkalinity into the inner (LCW) tubing;
stopping the DIC pump and allowing the solution within the LCW tubing to equilibrate, such that, when equilibrated, internal and external partial pressures of CO₂ are equal, R is constant, and, therefore, pH is constant;
calculating DIC based on pH and TA parameters.

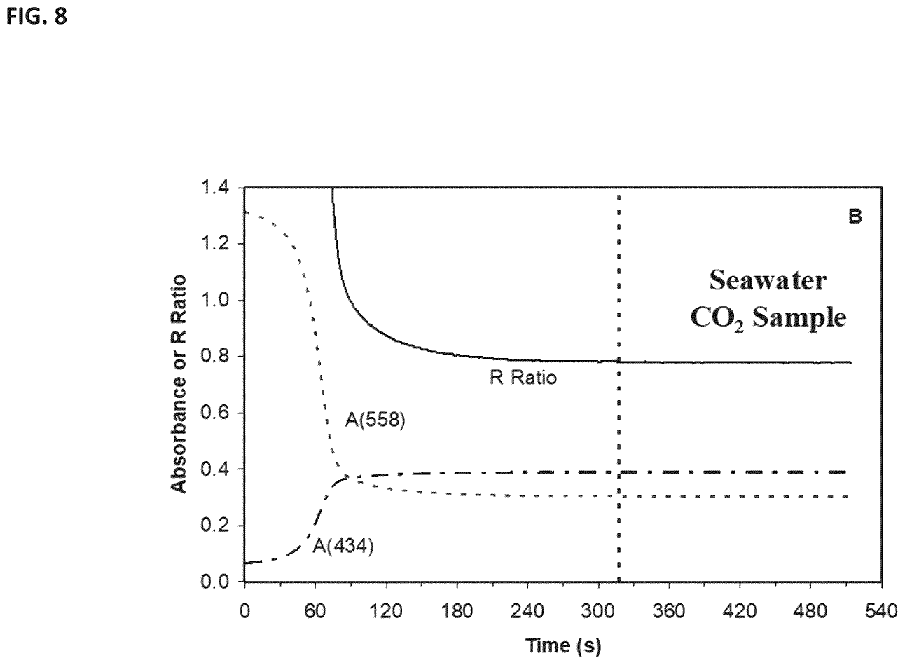

To this end, FIG. 8 provides an illustrative example of DIC analysis of a water sample according to many embodiments. It should be noted here that, the approach to the DIC quantification of many embodiments described herein is inherently antibiofouling and offers long term stability once B (t) is defined. In many embodiments, the DIC quantification method described herein is optimized for 50 readings per hour.

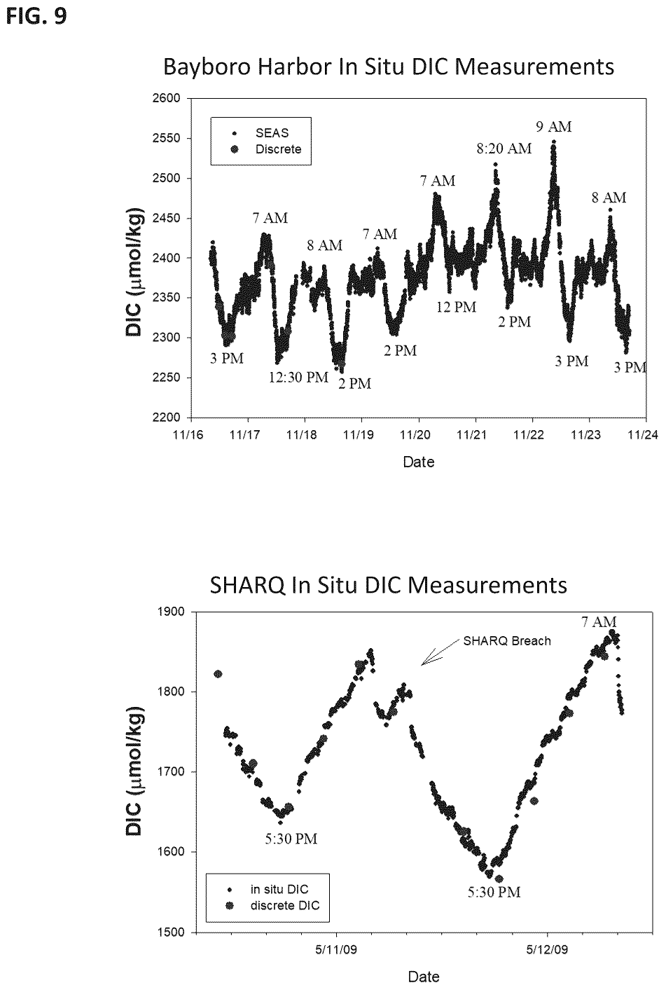

In addition, FIG. 9 provides examples of data collected via more conventional approaches to DIC quantification in a seawater sample, including Bayboro Harbor in situ DIC measurements method (top), and SHARQ sampling area in situ DIC measurements method. Notably, Bayboro Harbor in situ DIC measurements offer:
on average, 150 mM DIC range over a daily (diel) cycle, and
good agreement between in situ and discrete measurements, but
rapid changes, which illustrate need for high-resolution monitoring;
while SHARQ in situ DIC measurements offer:
on average, 250 mM DIC range over a daily cycle,
prevention of exchange with ambient water reduced noise in DIC signal, but
high resolution measurements needed to capture variability.
   Nevertheless, in many embodiments, the spectroscopic approaches to DIC quantification have inherent antifouling properties and, as such, can be used in high productivity areas. In addition, in these methods, as the sample passes through outside the light path, low particulate matter does not affect measurements.

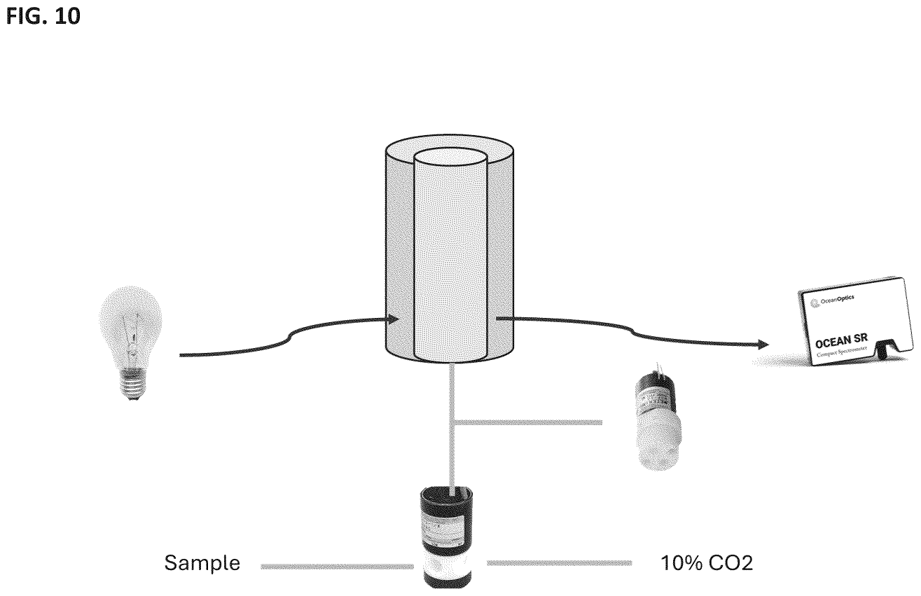

Furthermore, FIG. 10 schematically illustrates the TA channel employed in the device and method of the instant application for TA measurements. More specifically, and according to many embodiments, the TA channel comprises the TA optical cell, contained within an outer TA tubing, in optical communication with the light source and the TA spectrometer via an optical fiber; and in fluid communication with the sample selection valve, the TA dye reservoir, and a CO₂ supply of a known CO₂ concentration; and wherein the outer TA tubing is also in fluid communication with the CO₂ supply. In many embodiments, the optical cell comprises PEEK tubing. In many embodiments, the known CO₂ concentration of the CO₂ supply is 8 to 20%. In many embodiments, the known CO₂ concentration is 10%.

Accordingly, in many embodiments, and, as also, in some aspects, suggested by Fleger et al., Total alkalinity measurements in small samples: methods based on CO₂ equilibration and spectrophotometric pH. Analytica Chimica Acta (2025), the disclosure of which is incorporated herein by reference, the procedure to measure TA with the TA channel of many embodiments comprises:
with the sample/CO₂ selector on the sample side, turning on the sample pump, and flushing the TA optical cell with the sample;
taking a reference measurement;
pushing a plug of the TA dye into the TA optical cell loop before the sample;
switching the 3 way sample/CO₂ valve to CO₂ side, such as to carry the TA dye into the TA optical cell, and starting the equilibration with the known CO₂ gas in the outer tubing.

Accordingly, in many embodiments, the approach to solving the seawater inorganic carbon system is reversed from the DIC analysis, wherein, pCO₂ is kept constant instead of, more conventionally, TA. As such, in many embodiments, in direct purge TA analysis (A_{T}), the CO₂ supply of the known CO₂ concentration (e.g., 10%) is used to equilibrate the TA optical cell with seawater, wherein the equilibration is monitored with the TA dye (e.g., BCP) (FIG. 10). Knowing the pH of a seawater sample that has a known pCO₂ from the reference gas equilibration allows for the calculation of the sample's AT. Accordingly, Total Alkalinity (AT) of the sample is then calculated using the equation:

$$log ( {A}_{T} + [ {H}^{+} ] ) = log ( {( {K}_{0} )}_{i} ( ρ {CO}_{2} ) / {K}_{1} ) + E + pH$$

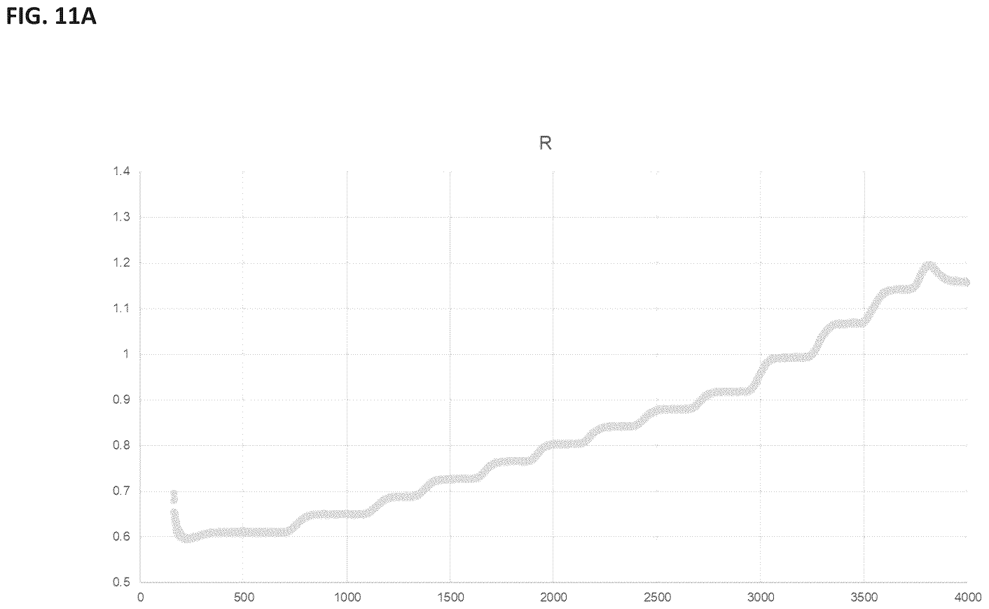

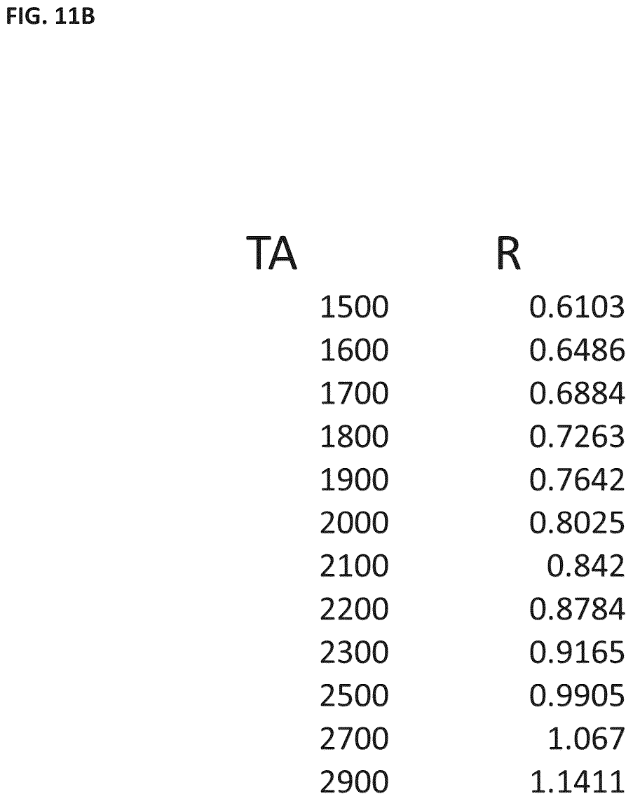

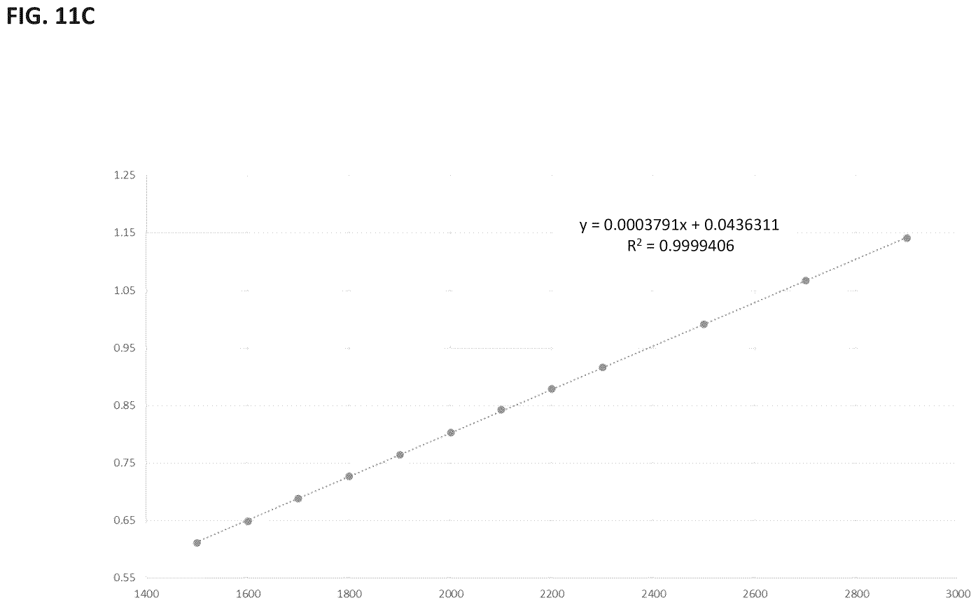

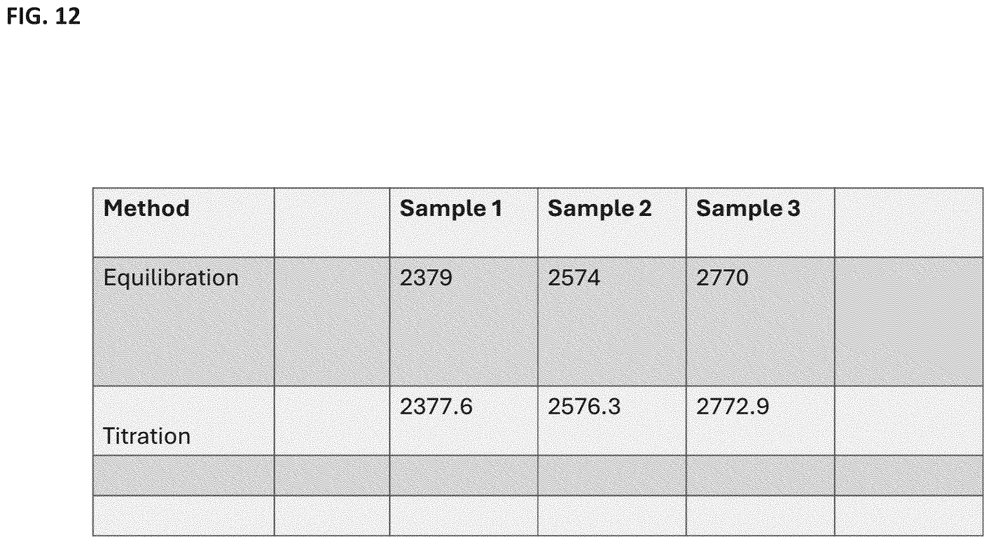

To this end, FIGS. 11A through 11C illustrate the various aspects of the TA analysis of a seawater sample with the TA channel according to many embodiments, while FIG. 12 compares the instant TA measuring approach based on equilibration to a more conventional titration approach.

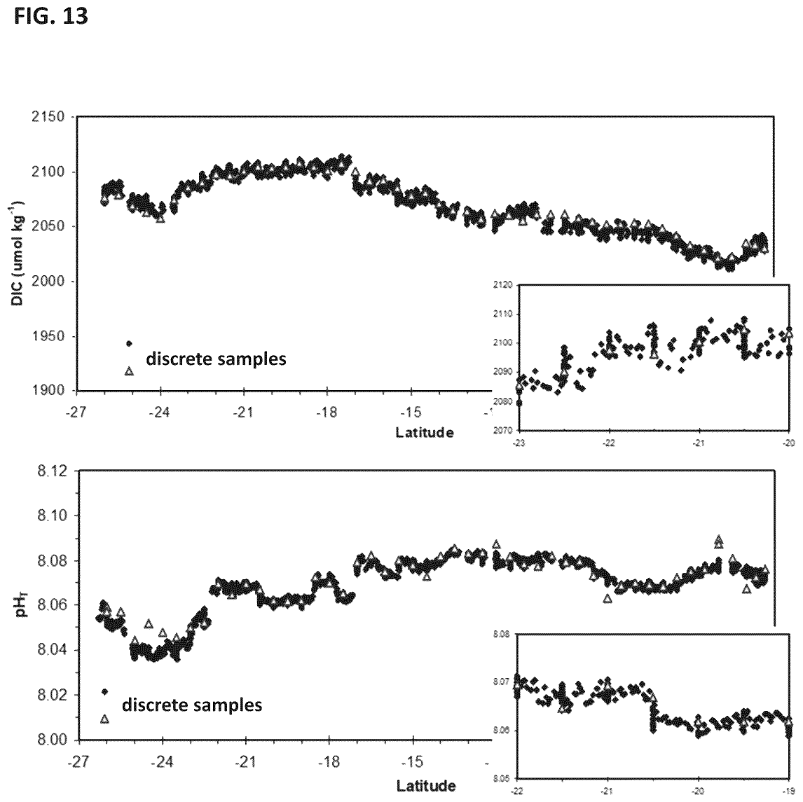

To this end, FIG. 13 provides illustrative data collected over a month of seawater sampling with the pH and DIC channels of the device and method of the instant application according to many embodiments. In many embodiments, the precision of the pH measurement is 0.001, the precision of DIC measurements is 2 μM/kg, and the precision of the TA measurement is 2 μM/kg.

In many embodiments, the design features of the device described herein allow for substantial miniaturization of the device. As such, in many embodiments, the device requires small sample sizes, wherein all 3 parameters can be measured on just milliliters of seawater samples. In many embodiments, the device fits in the enclosure that measures less than 6×2 ft², and less than 2×1.5 ft². In many embodiments, the enclosure is 22×17×11 inches or smaller.

It should be noted here that, although this application describes many embodiments wherein the samples subjected to the measurements by the device and method of the instant application are seawater samples, in some embodiments, the instant device and method are employed to characterize freshwater samples, such as, for example, but not limited to: water samples from lakes, rivers, streams, ground water, ponds, aquifers, and wells. In many such embodiments, the device and method utilize pH sensitive dyes that are distinct from the dyes specifically tuned to seawater pH ranges, but, otherwise, the device and method are applicable to freshwater samples according to the same principles.

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

What is claimed is:

1. A device for determining inorganic carbon system parameters for a pair of aqueous samples comprising:
an enclosure characterized by a footprint size further comprising:
a dry electronics chamber comprising a power supply, a computer, a display, a light source, a relay board, a connectivity hub, a square wave generator for pumps, a pH spectrometer, a DIC spectrometer, and a TA spectrometer;
a pumps and valves chamber comprising a plurality of diaphragm pumps further comprising a sample pump, a pH channel pump, a pH dye pump, a DIC channel pump, a DIC reference pump, a DIC dye pump, a DIC standard solution pump, TA channel pump, and a TA dye pump; and a plurality of valves further comprising: a sample selection valve, wherein the sample selection valve is a 3-way valve; a sample outlet, a 3-way TA sample valve, a 3-way CO₂ valve, and a 2-way DIC LCW shutoff valve;
a reagents chamber comprising a pH dye reservoir containing a pH dye; a DIC reservoir containing DIC reagents, including a DIC reference, and a DIC dye; a TA dye reservoir containing a TA dye; and a DIC acid reservoir containing a DIC acid;
a sample reservoir and
a wet chamber characterized by a temperature controlled by a temperature controller, further comprising
a pH channel comprising a pH optical cell in optical communication with the pH spectrometer and the light source, in thermal contact with the temperature controller, and in fluid communication with the sample selection valve, the sample outlet, and the pH dye reservoir;
a DIC channel comprising a DIC optical cell in optical communication with the DIC spectrometer and the light source, in thermal contact with the temperature controller, and in fluid communication with the sample selection valve, the sample outlet, the DIC acid reservoir, and the DIC reservoir;
a TA channel comprising a TA optical cell in optical communication with the TA spectrometer and the light source, in thermal contact with the temperature controller, and in fluid communication with the sample selection valve, the sample outlet, the TA dye reservoir, and a TA CO₂ supply of a known CO₂ concentration; and wherein
the temperature controller is disposed in an immediate proximity to the wet chamber to control the temperature.

2. The device of claim 1, wherein the wet chamber further comprises at least one or all of: a salinity sensor, a temperature sensor, and a stirring device to promote water circulation within the wet chamber.

3. The device of claim 1, wherein the temperature controller is a Peltier thermostat.

4. The device of claim 1, wherein the light source is an LED.

5. The device of claim 1, wherein the connectivity hub is a USB hub.

6. The device of claim 1, wherein the DIC optical cell comprises an LCW tubing enclosed within an outer PEEK tubing, such that the LCW tubing is in optical communication with the DIC spectrometer and the light source, and in fluid communication with the DIC reagent reservoir; while the outer PEEK tubing is in fluid communication with the sample selection valve, the sample outlet, and the DIC acid reservoir, such that the outer PEEK tubing contains a solution that is in diffusive communication with the LCW tubing.

7. The device of claim 6, wherein the LCW tubing is gas permeable, optically clear and is characterized by a refractive index of near 1.29 or less.

8. The device of claim 7, wherein the LCW tubing comprises Teflon AF.

9. The device of claim 1, wherein the TA channel comprises the TA optical cell enclosed within an outer TA tubing, such that the TA optical cell is in optical communication with the TA spectrometer and the light source; and in fluid communication with the sample selection valve, the TA dye reservoir, and the CO₂ supply; and wherein the outer TA tubing is in fluid communication with the CO₂ supply.

10. The device of claim 9, wherein the known CO₂ concentration of the CO₂ supply to the TA channel is 8 to 20%.

11. The device of claim 10, wherein the known CO₂ concentration is 10%.

12. A method for determining inorganic carbon system parameters comprising:
providing a device comprising:
an enclosure characterized by a footprint size further comprising:
a dry electronics chamber comprising a power supply, a computer, a display, a light source, a relay board, a connectivity hub, a square wave generator for pumps, a pH spectrometer, a DIC spectrometer, and a TA spectrometer;
a pumps and valves chamber comprising a plurality of diaphragm pumps further comprising a sample pump, a pH channel pump, a pH dye pump, a DIC channel pump, a DIC reference pump, a DIC dye pump, a DIC standard solution pump, TA channel pump, and a TA dye pump; and a plurality of valves further comprising: a sample selection valve, wherein the sample selection valve is a 3-way valve; a sample outlet, a 3-way TA sample valve, a 3-way CO₂ valve, and a 2-way DIC LCW shutoff valve;
a reagents chamber comprising a pH dye reservoir containing a pH dye; a DIC reservoir containing DIC reagents, including a DIC reference and a DIC dye; a TA dye reservoir containing a TA dye; and a DIC acid reservoir containing a DIC acid;
and a sample reservoir; and
a wet chamber characterized by a temperature controlled by a temperature controller, further comprising
a pH channel comprising a pH optical cell in optical communication with the pH spectrometer and the light source, in thermal contact with the temperature controller, and in fluid communication with the sample selection valve, the sample outlet, and the pH dye reservoir;
a DIC channel comprising a DIC optical cell in optical communication with the DIC spectrometer and the light source, in thermal contact with the temperature controller, and in fluid communication with the sample selection valve, the sample outlet, the DIC reservoir, and the DIC acid reservoir;
a TA channel comprising a TA optical cell in optical communication with the TA spectrometer and the light source, in thermal contact with the temperature controller, and in fluid communication with the sample selection valve, the sample outlet, the TA dye reservoir, and a TA CO₂ supply of a known CO₂ concentration; and wherein
the temperature controller is disposed in an immediate proximity to the wet chamber to control the temperature;
providing a pair of different aqueous samples comprising a first sample and a second sample;
splitting the first and the second samples into three aliquots each to obtain a first through a third aliquot of the first sample and a first through a third aliquot of the second sample;
feeding the first aliquot of the first sample through the sample selection valve to one of: the pH channel, the DIC channel, or the TA channel;
measuring a first parameter, wherein the first parameter is a parameter corresponding to the selected channel, that is a first pH, a first DIC, or a first TA, and
purging the first aliquot of the first sample from the device through the sample outlet;
feeding the first aliquot of the second sample through the sample selection valve to the same channel,
measuring a second parameter, wherein the second parameter is a parameter corresponding to the selected channel, that is a second pH, a second DIC, or a second TA, and
purging the first aliquot of the second sample from the device through the sample outlet;
recording the first parameter, the second parameter, and
calculating the difference between the first parameter and the second parameter;
repeating feeding, measuring, purging, recording, and calculating steps for the remaining aliquots for the remaining channels,
to completely characterize the inorganic carbon system parameters of the first and the second samples.

13. The method of claim 12, wherein the wet chamber further comprises at least one or all of: a salinity sensor, a temperature sensor, and a stirring device to promote water circulation within the wet chamber.

14. The method of claim 12, wherein the temperature controller is a Peltier thermostat.

15. The method of claim 12, wherein the light source is an LED.

16. The method of claim 12, wherein the aqueous component of the pair of different aqueous samples is seawater.

17. The method of claim 12, wherein the aqueous component of the pair of different aqueous samples is freshwater.

18. The method of claim 12, wherein the DIC optical cell comprises an LCW tubing enclosed within an outer PEEK tubing, such that the LCW tubing is in optical communication with the DIC spectrometer and the light source, and in fluid communication with the DIC reagent reservoir; while the outer PEEK tubing is in fluid communication with the sample selection valve, the sample outlet, and the DIC acid reservoir, such that the outer PEEK tubing contains a solution that is in diffusive communication with the LCW tubing.

19. The method of claim 18, wherein the LCW tubing is gas permeable, optically clear and is characterized by a refractive index of near 1.29 or less.

20. The method of claim 12, wherein the TA channel comprises the TA optical cell enclosed within an outer TA tubing, such that the TA optical cell is in optical communication with the TA spectrometer and the light source; and in fluid communication with the sample selection valve, the TA dye reservoir, and the CO₂ supply; and wherein the outer TA tubing is in fluid communication with the CO₂ supply.

21. The method of claim 18, wherein the known CO₂ concentration of the CO₂ supply to the TA channel is 10%.

22. The method of claim 18, wherein measuring the first and the second DIC, each, comprises:
flushing the outer PEEK tubing with the first or second sample;
acidifying the first or second sample with the DIC acid;
flushing the LCW tubing with the DIC reference;
taking a reference measurement;
pumping the DIC dye solution of known alkalinity into the LCW tubing; and
allowing to equilibrate prior to taking a measurement.

23. The method of claim 18, wherein measuring the first and the second TA, each, comprises:
flushing the TA optical cell with the first or second sample;
taking a reference measurement;
pushing a plug of the TA dye in the TA channel loop just prior to the optical cell;
using the CO₂ supply to carry the TA dye into the TA optical cell, and
allowing to equilibrate with the CO₂ gas in the outer PEEK tubing prior to taking a measurement.
